(19) United States
(12) Patent Application Publication
(10) Pub. No.: US 2013/0218207 A1
(43) Pub. Date: Aug. 22, 2013

(54) DYNAMIC MULTI-AXIAL ANCHOR

(71) Applicants: Thomas A. Carls, Memphis, TN (US); Christopher Shaffrey, Charlottesville, VA (US)

(72) Inventors: Thomas A. Carls, Memphis, TN (US); Christopher Shaffrey, Charlottesville, VA (US)

(73) Assignee: WARSAW ORTHOPEDIC, INC, Warsaw, IN (US)

(21) Appl. No.: 13/398,550

(22) Filed: Feb. 16, 2012

(51) Int. Cl. A61B 17/70; A61B 17/88

(52) U.S. Cl. 606/278; 606/279

(57) ABSTRACT

A device for dynamically attaching an elongated member to a vertebral member. The device may include a receiver with a channel configured to receive an elongated member. A fastener may secure the elongated member to the receiver. The receiver may further include a reservoir sized to receive a head of an anchor. The reservoir may be shaped for movement of the receiver relative to the anchor within one plane. This movement may accommodate spinal motion during flexion and extension of the patient. One or more dampeners may be positioned in the reservoir and contact against the anchor during the movement. The dampeners may provide resistance to the movement of the patient.

### BACKGROUND

The present application is directed to an anchoring device with an anchor and a receiver for attaching an elongated construct to a vertebral member and, more particularly, to an anchoring device configured for dampened movement of a receiver relative to an anchor.

The spine is divided into a variety of regions including the cervical, thoracic, and lumbar regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebrae that form the sacrum and the coccyx.

Various conditions may lead to damage of the intervertebral discs and the vertebral members. The damage my result from a variety of causes including a specific event such as trauma, a degenerative condition, a tumor, or infection. Damage to the intervertebral discs and vertebral members can lead to pain, neurological deficit, and/or loss of motion.

Constructs, such as vertebral rods and associated anchors, may provide a stable, rigid column for various treatments of the vertebral members. The constructs may be used to encourage fusion between vertebral members after spinal-fusion surgery. Further, the constructs may redirect stresses over a wider area away from a damaged or defective region. Also, the constructs may restore the spine to its proper alignment. In the various surgical procedures, the constructs may be attached to the exterior of two or more vertebral members, whether it is at a posterior, anterior, or lateral side of the spine.

Proximal Junctional Kyphosis (PJK) is a hyperkyphosis of a vertebral segment immediately proximal to a spinal construct. PJK may result in facet dislocation and posterior widening of the disc space. Constructs may be designed to prevent or reduce the occurrence of PJK.

### SUMMARY

One embodiment is directed to a device for attaching an elongated member to a vertebral member. The device includes an anchor with a head and an outwardly-extending shaft. A receiver is attached to the anchor and includes a channel configured to receive the elongated member and a reservoir configured to receive the head of the anchor. The receiver has a longitudinal axis that extends through the channel and the reservoir. The reservoir includes an elongated shape with a major axis that extends between opposing first and second ends and a minor axis. The reservoir is larger than the head of the anchor with the receiver movable about the head at a plurality of positions. A first dampener is positioned in the reservoir between the first end and the head of the anchor to dampen movement of the receiver relative to the anchor in a first direction. A second dampener is positioned in the reservoir between the second end and the head of the anchor to dampen movement of the receiver relative to the anchor in an opposing second direction. The second dampener is separate from and spaced away from the first dampener.

The first dampener may include a first construction and the second dampener may include a different second construction. The first dampener may include a greater thickness measured along the major axis than the second dampener. The receiver may also be constructed with a first distance in the reservoir between the longitudinal axis and the first end is greater than a second distance between the longitudinal axis and the second end. The reservoir may further include an arced shape in a plane that bisects the channel with a central region of the reservoir being closer to the channel than each of the first and second ends. The receiver may include a neck that forms a reduced opening in communication with the reservoir with the opening having a smaller width than the head of the anchor. The device may also include a fastener that engages with the receiver and applies a downward force to the elongated member into the channel with the head of the anchor being spaced away and isolated from the channel with the head being isolated from the force. The device may be constructed with the reservoir sized relative to the head of the anchor for the receiver to be rotatable about the anchor. The device may also include an opening within an interior of the receiver that extends between the channel and the reservoir with the opening including a smaller width than the head of the anchor.

Another embodiment is directed to a device for attaching an elongated member to a vertebral member. The device includes an anchor with an enlarged head and an outwardly-extending shaft A receiver includes a reservoir that receives the head of the anchor with the reservoir being larger than the head of the anchor for the receiver to be movable to a plurality of positions relative to the anchor. The receiver further includes a channel configured to receive the elongated member. The receiver has a longitudinal axis that extends through the channel and the reservoir. The reservoir includes an elongated curved shape in a plane that extends through the channel with first and second ends positioned a greater distance from the channel than a central section of the reservoir. A flexible dampener is positioned in the reservoir and positioned between the first end of the reservoir and the head of the anchor. The dampener is positioned to be compressed when the receiver moves in a first direction relative to the anchor.

An additional dampener may be positioned in the reservoir between the second end and the head of the anchor and is compressed when the receiver moves in an opposing second direction relative to the anchor. The additional dampener may be separate from and spaced away from the dampener. The additional dampener may include a different flexibility than the dampener. A first distance between the longitudinal axis and the first end of the reservoir may be greater than a second distance between the longitudinal axis and the second end of the reservoir. The receiver may include a narrow neck that defines an opening that extends into the reservoir and includes a smaller width than the head of the anchor. The device may further include a fastener that engages with the receiver and applies a downward force to the elongated member into the channel with the head of the anchor being spaced away from the channel with the head being isolated from the force. The reservoir may be sized relative to the head of the anchor for the receiver to be rotatable about the anchor. An opening may be located within an interior of the anchor that extends between the channel and the reservoir with the opening including a smatter width than the head of the anchor.

A method of stabilizing a patient's spine is also disclosed. The method includes attaching a dynamic device to a vertebral member; aligning a receiver of the dynamic device relative to the vertebral member with a major axis of a reservoir in the receiver aligned in a sagittal plane of the patient's spine with a first dampener positioned on a first side of an anchor head and a second dampener positioned on a second side of the anchor head. The first and second dampeners are spaced apart and separate. The method includes positioning an elongated member into a channel of the receiver; attaching a fastener to the receiver and positioning the elongated member against a bottom of the channel with the receiver being movable relative to the anchor. The dynamic device is configured for the receiver to move relative to the anchor in a first direction in the sagittal plane to compress the first dampener and to move in an opposing second direction in the sagittal plane to compress the second dampener.

The method may also include positioning the dynamic device for the receiver to rotate about the anchor head. The method may also include positioning the dynamic device to limit movement of the receiver in the first direction to about 3-4° and to limit movement of the receiver in the second direction to about 6-8°.

The various aspects of the various embodiments may be used alone or in any combination, as is desired.

### BRIEF DESCRIPTION OF THE DRAWINGS

FIG. 1 is a schematic cross-sectional view of a device attaching a elongated member to a vertebral member.

FIG. 2 is a perspective view of a device attaching an elongated member to a vertebral member.

FIG. 3 is an exploded view of a device with an elongated member.

FIG. 4 is a sectional view cut along line IV-IV of FIG. 2.

FIG. 5 is a sectional view cut along line V-V of FIG. 2 of one embodiment of the reservoir and first and second dampeners.

FIG. 6 is a sectional view cut along line V-V of FIG. 2 of another embodiment of the reservoir and first and second dampeners.

FIG. 7 is a sectional view cut along line V-V of FIG. 2 of another embodiment of the reservoir and a dampener.

FIG. 8 is a sectional view cut along line V-V of FIG. 2 of another embodiment of the reservoir and first and second dampeners.

FIG. 9 is a schematic side view of a spinal construct including a dynamic device attached along a spinal segment.

### DETAILED DESCRIPTION

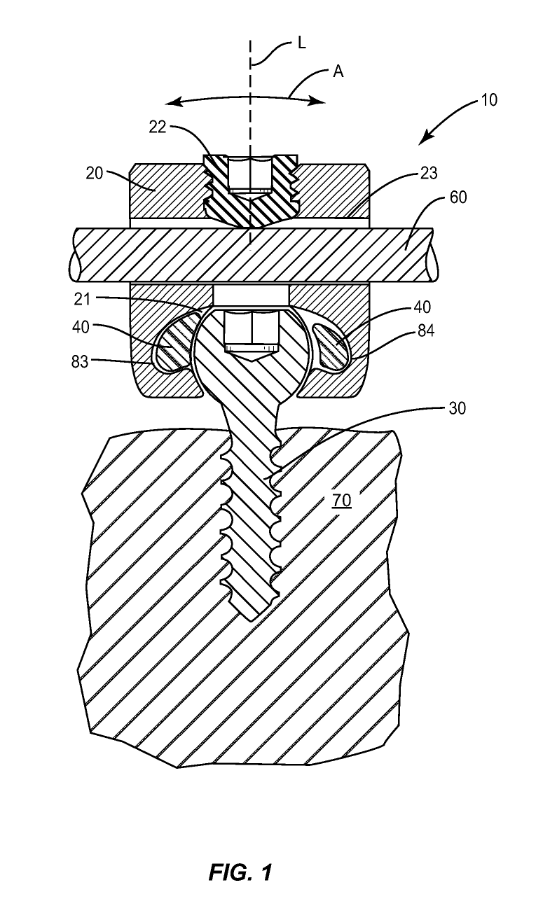

The present application is directed to a device for dynamically attaching an elongated member to a vertebral member. As illustrated schematically in FIG. 1, the device 10 includes a receiver 20 with a channel 23 configured to receive an elongated member 60. A fastener 22 secures the elongated member 60 to the receiver 20. The receiver 20 further includes a reservoir 21 that receives a head of an anchor 30. The reservoir 21 is shaped for movement of the receiver 20 relative to the anchor 30 within one or more planes. The movement may include pivoting movement and sliding movement of the receiver 20 relative to the anchor 30. This movement may accommodate spinal motion, such as that illustrated by arrow A which may occur during flexion and extension of the patient, as well as various other movements. One or more dampeners 40 are positioned in the reservoir 21 and contact against the anchor 30 during the movement. The dampeners 40 provide resistance to the movement.

The device 10 is configured to dynamically attach the elongated member 60 to the vertebral member 70. The device 10 may be configured for the receiver 20 to pivot about the anchor head 31 fir movement along an arc. The device 10 may also be configured for the receiver 20 to move in a sliding motion relative to the anchor 30. Further, the device 10 may provide for rotational movement of the receiver 20 about the anchor 30, and for axial movement along the axis of the anchor 30. The device 10 may provide for just a single type of motion, or may provide for two or more types of motion.

The device 10 may be positioned in the patient and configured for movement in one or more planes (e.g., sagittal, coronal, axial planes). In one embodiment, the device 10 is configured for dynamic movement in a single plane with movement being prevented in the other planes. In one embodiment, the device 10 is configured for movement in two planes with movement prevented in a third plane. Various combinations of movements and planes may be achieved by the device 10 depending upon the needs of the patient.

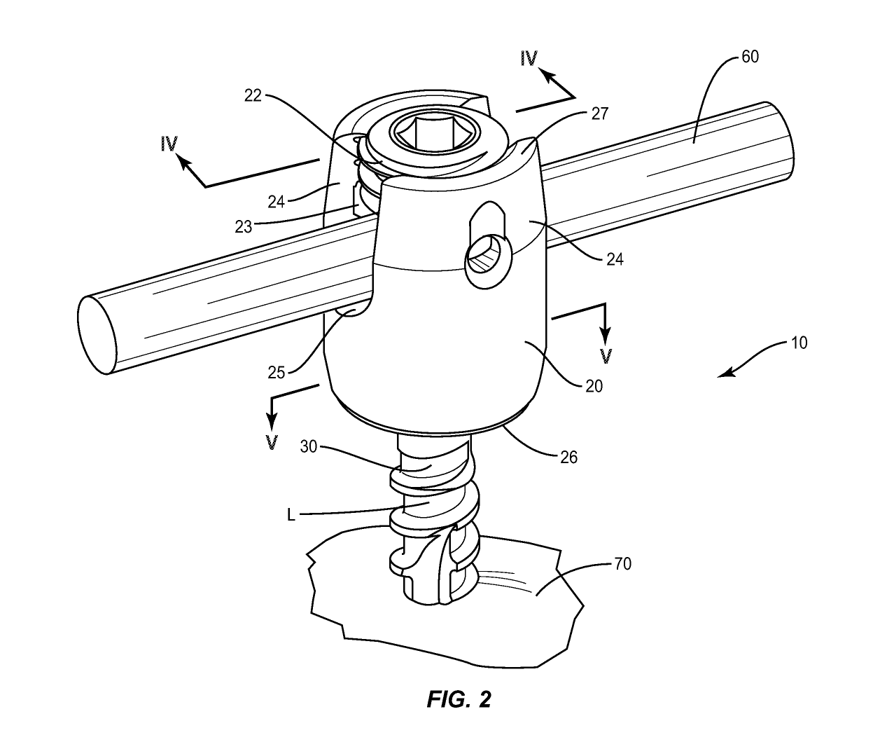

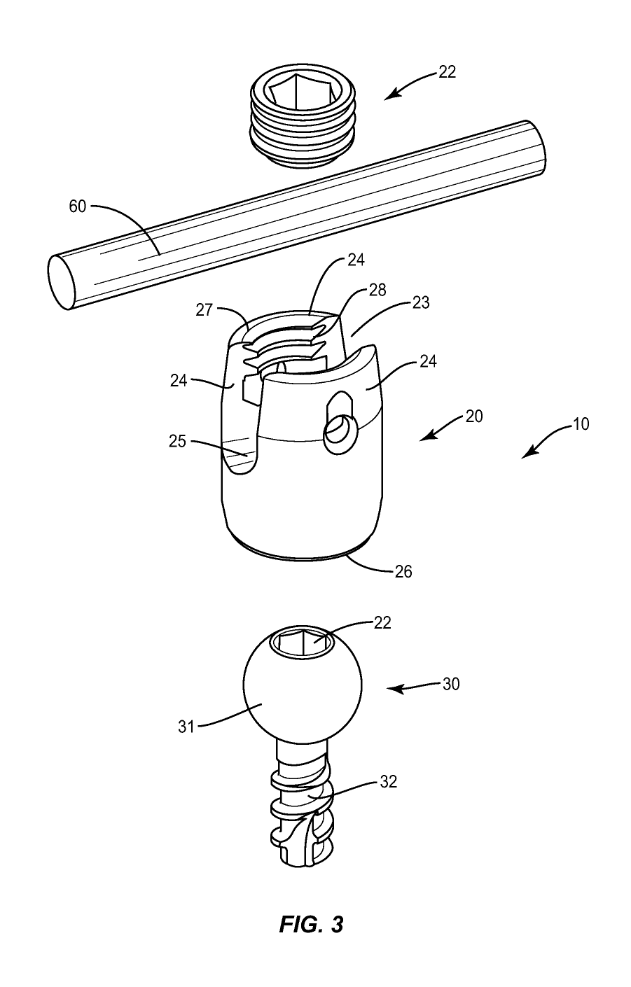

The receiver 20 is configured to receive both the elongated member 60 and the anchor 30. As illustrated in FIGS. 2, 3, and 4, the receiver 20 includes a top side 27 and a bottom side 26. The top side 27 faces away from the vertebral member 70 when the receiver 20 is implanted into the patient and the bottom side 26 faces towards the vertebral member 70. A longitudinal axis L of the receiver 20 extends through the channel 23 and the reservoir 21.

The channel 23 is formed between a pair of opposing arms 24 and extends into the top side 27 of the receiver 20. The channel 23 is open on the top side 27 and includes an opposing bottom 25. The bottom 25 may include a rounded shape to conform to the shape of the elongated member 60. The bottom 25 may also include various other shapes that correspond to the shape of the elongated member 60. Threads 28 may be located along the arms 24 to receive a fastener 22 to secure the elongated member 60 within the channel 23. The threads 28 may be located along the interior of the arms 24 as illustrated, or may be located along the exterior of the arms 24 depending upon the type of fastener 22.

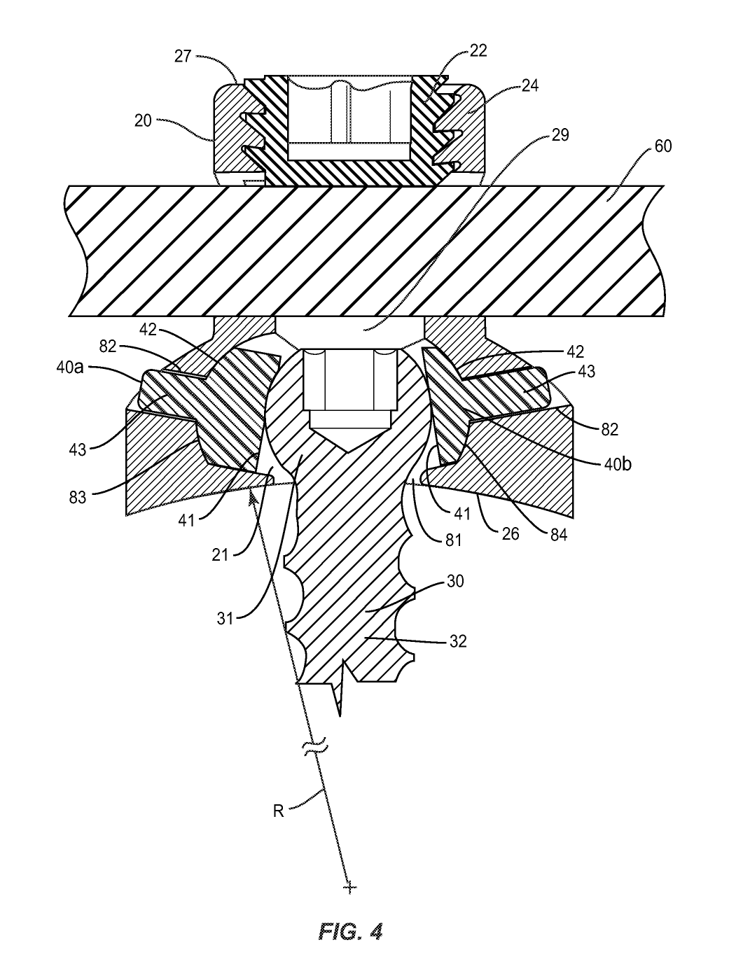

The bottom side 26 of the receiver 20 may be curved to facilitate rotation of the receiver 20 about the anchor 30 and accommodate the biomechanical motion of the spine. As illustrated in FIG. 4, the bottom side 26 bows upward in a central section away from the outer ends. In one embodiment, the bottom side 26 includes a substantially constant radius R. Other embodiments include a radius along a limited section of the bottom side 26.

The receiver 20 further includes a reservoir 21 to receive the head 31 of the anchor 30. As illustrated in FIG. 4, the reservoir 21 extends through the bottom side 26 of the receiver 20 and is spaced away from the bottom side 25 of the channel 23. The reservoir 21 is larger than the anchor head 31 to allow movement of the receiver 20 relative to the anchor 30 during movement of the patient as will be explained in more detail below. The reservoir 21 also includes a narrow neck 81 at or in proximity to the bottom side 26 of the receiver 20. The neck 81 has a width that is smaller than the anchor head 31 to maintain the head within the interior of the reservoir 21.

The interior of the receiver 20 may be hollow with an opening 29 that extends between the bottom 25 of the channel 23 and the reservoir 21. The opening 29 is sized to receive a tool that is inserted through the top side 27 of the receiver 20 and is sized to engage the head 31 of the anchor 30 to attach the anchor 30 and the receiver 20 to the vertebral member 70. In another embodiment, the receiver 20 is solid between the bottom 25 of the channel 23 and the reservoir 21 (i.e., there is no opening 29).

Further, the reservoir 21 is spaced away and isolated from the channel 23. A force applied by the fastener 22 to the elongated member 60 is isolated from the anchor head 31. This spacing and isolation allow for the receiver 20 to move relative to the anchor 30 when the elongated member 60 is secured to the receiver 20.

The reservoir 21 is shaped to control the movement of the receiver 20 relative to the anchor 30. As illustrated in FIGS. 1 and 4, the reservoir 21 includes an elongated shape with opposing first and second ends 83, 84. In a plane illustrated in FIGS. 1 and 4, the reservoir 21 further includes a curved shape. The ends 83, 84 of the reservoir 21 are positioned farther away from the channel 23 than a central section of the reservoir 21. FIGS. 1 and 4 include embodiments with the reservoir 21 having a symmetrical shape and size about the longitudinal axis L. Other embodiments may include the reservoir 21 having a non-symmetrical shape with a first section of the reservoir 21 between the longitudinal axis L and first end 83 having a different shape and/or size than a second section between the longitudinal axis L and the second end 84. In one embodiment, the major axis of the reservoir 21 and the channel 23 are aligned.

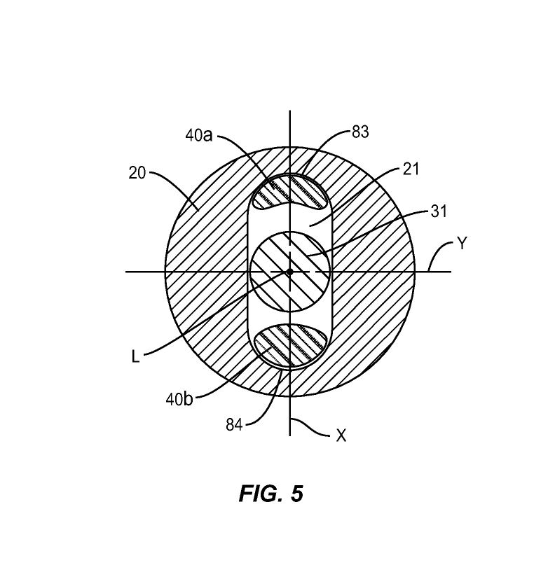

FIG. 5 illustrates a schematic view of the reservoir 21 in a second plane that is perpendicular to the longitudinal axis L. The reservoir 21 includes an extended length measured along the x axis that extends through the first and second ends 83, 84. The reservoir 21 further includes a narrow width measured along the y axis.

FIG. 5 includes the reservoir 21 having a uniform shape about the longitudinal axis L. A first distance between the longitudinal axis L and a first end 83 of the reservoir 21 is the same as a second distance between the longitudinal axis L and an opposing second end 84. An amount of travel along axis x is limited by the shape of the reservoir 21 defined by the ends 83, 84.

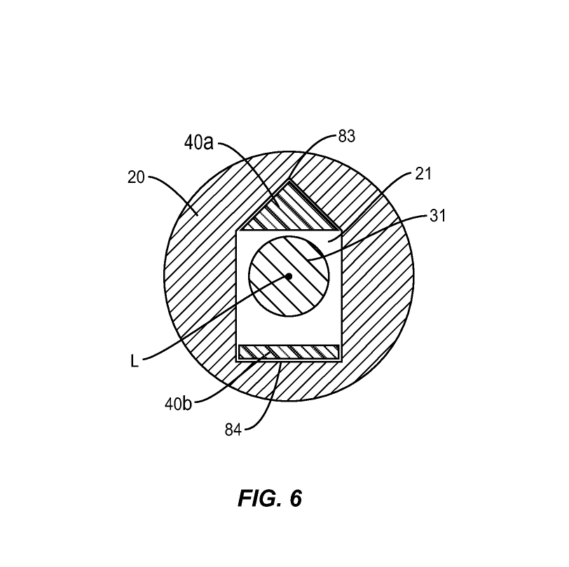

The reservoir 21 may further be shaped to allow additional movement in one direction relative to a second direction. FIG. 6 includes the reservoir 21 with the longitudinal axis L located a greater distance away from the first end 83 than from the second end 84. This sizing difference allows for a greater amount of movement of the receiver 20 relative to the anchor head 31 in a first direction than in a second direction.

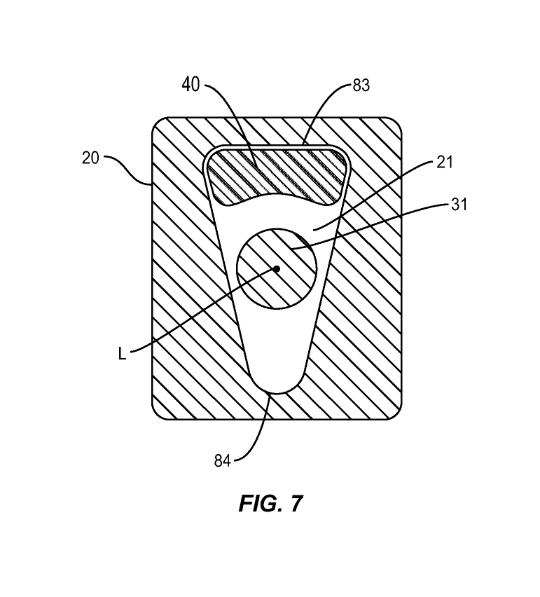

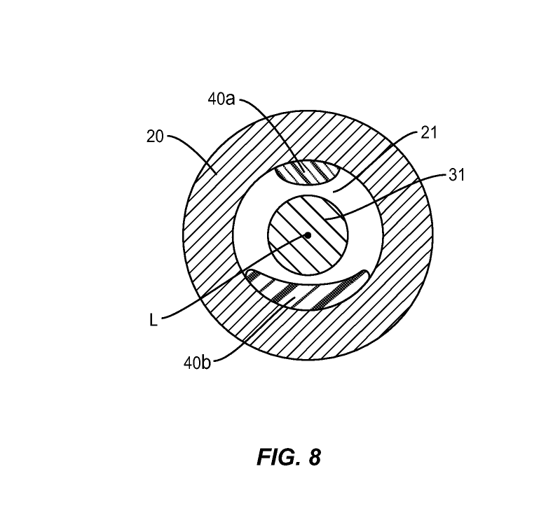

The reservoir 21 may also include various widths. In one embodiment, the width is the same along the entire length of the reservoir 21. FIG. 5 includes the width of the reservoir tapering at each of the ends 83, 84. FIG. 6 includes a tapered width at the first end 83 and a constant width at the second end 84. FIG. 7 includes a width that tapers from the first end 83 to the second end 84. FIG. 8 includes a reservoir 21 with a circular sectional shape.

The anchor 30 includes a head 31 that fits within the reservoir 21 and a shaft 32 configured to engage with the vertebral member 70. The head 31 is wider than the shaft 32. This difference in size provides for the head 31 to be maintained in the reservoir 21 with the shaft 32 sized to extend through the opening formed by the relatively narrow neck 81 of the receiver 20. The shaft 32 may have various shapes and configurations, including being threaded as illustrated in FIGS. 2, 3, and 4 to engage with the vertebral member 70, and having a hook-shape to engage with the vertebral member 70.

The relative shapes and sizes of the reservoir 21 and the anchor head 31 may provide for one or more different types of movement. The movements may include one or more of pivoting movement, sliding movement, rotational movement, and axial movement. The pivoting and sliding movement may be limited to just along the major axis of the reservoir 21, or may also provide for movement transverse to the major axis. The rotational movement provides for rotational movement of the receiver 20 about the anchor 30. Axial movement provides for movement of the receiver 20 along the axis of the anchor 30 (i.e., into and out of the patient). Different embodiments may provide for a single type or for multiple types of movement.

The sides of the reservoir 21 limit the extent of movement of the receiver 20 relative to the anchor 30 in the various directions. The sides of the reservoir 21 act as stop limits to control the movements. Contact between the head 31 and the sides prevent further movement in the various directions.

One or more dampeners 40 are positioned within the reservoir 21 to further control the movement of the receiver 20 relative to the anchor 30. The one or more dampeners 40 are positioned between the sides of the reservoir 21 and the head 31 to further control the movement in the various directions. The dampeners 40 may be constructed to dampen the movement in a particular direction, but still allow for additional movement until the head 31 contacts the side of the reservoir 21. The dampeners 40 may further be constructed to act as stop limits to stop further movement in a particular direction prior to the head 31 contacting the side of the reservoir 21.

The dampeners 40 may be made of a pliable polymer, such as a soft polyurethane composition or a silicone composition. Alternatively, the dampeners 40 may be made from a semi-rigid material such as PEEK, flexible polyurethane or polypropylene. Further, the dampeners 40 may be made from a rigid material, such as, for example, medical grade stainless steel, titanium, a titanium alloy or other metallic alloy, and/or a nonmetallic composition that is formed into a spring. The softer materials provide for more motion of receiver 20 relative to the anchor 30. Similarly, a semi-rigid material would provide a lesser amount of motion of the receiver 20 relative to the anchor 30.

The dampeners 40 are sized to fit within the reservoir 21 and contact against the sides of the reservoir 21 and the anchor head 31. As illustrated in FIG. 4, the dampeners 40 include an inner side 41 that faces towards the anchor head 31. The inner side 41 may be shaped to accommodate the shape of the anchor head 31. In one embodiment, the inner side 41 has a concave shape to receive the spherical anchor head 31. The inner side 41 may include various other shapes including but not limited to convex and planar. Dampeners 40 also include an outer side 42 that faces away from the anchor head 31. The outer side 42 may be shaped to match the configuration of the reservoir 21.

The dampeners 40 may be attached in the reservoirs 21 in a variety of different manners. The dampeners 40 may include mounts 43 that extend outward from the outer side 42 and attach with the receiver 20. In one embodiment, the receiver 20 includes openings 82 sized to receive the mounts 43. The openings 82 may extend through the side of the reservoir 21 such that the mounts 43 are exposed on the exterior of the receiver 20. Dampeners 40 may also be attached to the receiver by an adhesive or a mechanical fastener. In another embodiment, the dampeners 40 are compressed within the reservoir 21 to maintain attachment with the receiver 20.

The dampeners 40 may be configured to equally control the movement of the receiver 20 relative to the anchor 30 in two or more directions. The dampeners 40 may also be configured to allow different amounts of dampening for movement of the receiver 20 in different directions. The different amounts of dampening may be based on dampeners 40 of different construction, size, and/or shape. FIG. 4 includes an embodiment with a first dampener 40a being larger than an opposing second dampener 40b. This embodiment further includes the first dampener 40a having a different construction than the second dampener 40b. One or both of the differences in size and construction provides for different amounts of dampening of the receiver 20 in the opposing directions.

FIGS. 5, 6, and 8 each include an embodiment with a first dampener 40a having a different construction and a different shape than the opposing dampener 40b. These differences provide for different amount of dampening.

In embodiments with two or more dampeners 40, the dampeners 40 may be spaced away from each other.

The dampeners 40 may be positioned for controlling movement in one or more directions. The embodiments of FIGS. 4, 5, 6, and 8 provide for dampening of movement in two opposing directions. The dampeners 40 may be limited to specific sections of the reservoir 21. This allows for un-dampened movement of the receiver 20 in the various directions in which there is not a dampener 40 between the side of the reservoir 21 and the anchor head 31.

Some embodiments include a single dampener 40 that extends in a limited amount of the reservoir 21. FIG. 7 includes an embodiment with a single dampener 40. The dampener 40 is positioned at the first end 83 to dampen movement of the receiver 20 in a first direction relative to the anchor 30. The opposing second end 84 of the reservoir 21 does not include a dampener 40. Movement of the receiver 20 in other directions is not dampened and is only limited due to contact between the second end 84 and the anchor head 31.

In some embodiments, the one or more dampeners 40 extend a limited distance around the anchor head 31. Other embodiments may include the one or more dampeners 40 extending completely around the head of the anchor 30. A single dampener 40 may extend completely around the head 31, or two or more dampeners 40 in combination may extend completely around the head 31. The shape and size of the one or more dampeners 40 around the anchor head 31 may be substantially the same, or there may be variations. In one embodiment, the size of the one or more dampeners 40 is greater at the major axis of the reservoir 21, and smaller at points away from the major axis.

In addition to pivoting and sliding movement of the receiver 20 relative to the anchor 30, the receiver 20 may also be able to rotate about the anchor 30. The amount of rotation may provide for the receiver 20 to completely rotate about the anchor 30, or rotate just a limited amount about the anchor 30.

The elongated member 60 may be a spinal rod, plate, bar, or other elongated element having a length to extend between at least two vertebral members 70. The elongated member 60 may be solid or hollow along some or all of its length and/or may be of homogenous or heterogeneous composition. The elongated member 60 may be constructed from various materials, including but not limited to stainless steel, titanium, PEEK, and ceramic. The elongated member 60 may be substantially straight, or may be curved along the entire length, or along just a discrete section. The elongated member 60 may include various cross-sectional shapes including but not limited to circular, oval, and polygonal.

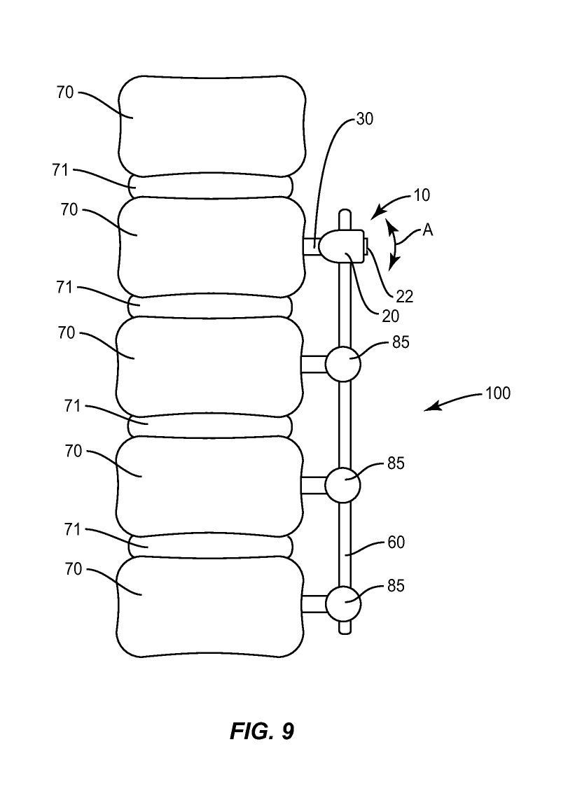

FIG. 9 illustrates one application of the device 10 in use within a patient to stabilize the spine. The device 10 is part of a spinal construct 100 that also includes other connectors 85 and an elongated member 60. In use, a surgeon attaches the other connectors 85 to vertebral members 70 along a length of the spine. Each of the other connectors 85 generally includes an anchor that is attached to a vertebral member and a receiver. The anchors and receivers of the other connectors 85 may be non-movably attached, or may be movably attached with the receiver positionable at a variety of angular positions. The device 10 is also attached to one of the vertebral members 70. In one embodiment as illustrated in FIG. 9, the device 10 is positioned closest to one of the ends of the elongated member 60. In one embodiment, the device 10 is positioned at the top spinal level of the construct 100. Other embodiments include the device 10 positioned at intermediate locations away from the ends of the elongated member 60 and inward from the other connectors 85.

The device 10 is positioned in the patient with the reservoir 21 aligned relative to the patient to allow for the desired movement. In this embodiment, the device 10 is positioned to allow movement indicated by arrow A.

The elongated member 60 is attached to each of the connectors 85 and the device 10. For the device 10, the elongated member 60 is inserted into the channel 23 and the fastener 22 is secured to the receiver 20 to capture the elongated member 60. The fastener 22 also secures the elongated member 60 against the bottom 25 of the channel 23 (see FIG. 2). The anchor head 31 is spaced away from the channel 23 and isolated from the force that is applied through the fastener 22 to the elongated member 60. This spacing and isolation enables the receiver 20 to move relative to the anchor 30.

In one embodiment, the device 10 is positioned in the patient to accommodate movement in the sagittal plane. The device 10 may be positioned with the major axis of the reservoir 21 aligned in the sagittal plane to allow movement of the receiver 20 during motion of the spine.

The length of the reservoir 21 and the one or more dampeners 40 dictate the extent of the movement. The reservoir 21 and one or more dampeners 40 may be configured to allow for different amounts of movement in the different directions. In one embodiment, the device 10 is configured to allow for about 3°-4′ of extension of the spine. In one embodiment, the device 10 is configured to allow for about 6°-8° of flexion of the spine. This movement is dampened in one or both directions by the one or more dampeners 40. In one embodiment, movement in the first direction is dampened as a first dampener 40a is compressed between the head 31 and the first end 83. Movement in the second direction is dampened by a second dampener 40b being compressed between the head 31 and the second end 84. The amount of dampening depends upon the construction and size of the dampeners 40. In embodiments with a single dampener 40, movement is dampened in just one direction.

The arced bottom side 26 of the receiver 20 and the arced shape of the reservoir further facilitates the movement of the receiver 20. The curved shapes accommodate the biomechanical motion of the spine.

The receiver 20 is further rotatable about the anchor head 31. Therefore, spinal movement may also cause the rotation of the receiver 20 about the anchor head 31.

FIG. 9 illustrates an embodiment with the device 10 configured for movement in the sagittal plane. The device 10 may also provide for movement in one or more of the coronal and axial planes. This embodiment includes one or more sliding and pivoting movement within the plane. The embodiment may also provide for rotational and/or axial movement relative to the anchor 30.

In one embodiment, the device 10 is positioned at an outer extent of the overall spinal construct. FIG. 9 illustrates the device 10 placed at the upper extent of the construct 100. This placement and the dynamic ability of the device 10 allows for spinal movement that may reduce or eliminate Proximal Junctional Kyphosis (PJK). PJK is a hyperkyphosis of the vertebral segment immediately proximal to a spinal construct. PJK occurs in long spinal constructs, and there are multiple factors that have been hypothesized to play a role in its development. A weakening of the muscles due to muscle dissection, the disruption of the posterior tension band, hybrid constructs (screw/hook), and pre-existing kyphotic hyperflexion have all been reasoned to play a role. Possible side effects of the condition might include facet dislocation and posterior widening of the disc space.

FIG. 9 illustrates a construct 100 that includes a single device 10. Other constructs include two or more devices 10. The devices 10 may be adjacent to each other along the elongated member 60, or may be spaced apart with other fasteners 85 between the devices 10. In one embodiment, the construct 100 includes devices 10 providing each of the connections between the vertebral members 70 and the elongated member 60 (i.e., there are no other types of fasteners). When multiple devices 10 are employed in a construct 100, the different devices 10 may have the same or different constructions. Further, the different devices 10 may be configured and/or positioned to provide for the same or different types and directions of movement.

The device 10 may be configured and positioned within the patient for movement in one or more of the sagittal, coronal, and axial planes. One example includes the device 10 being dynamically movable within one or more the planes and fixed within the other planes. One specific example includes a device 10 movable in the sagittal and coronal planes and fixed in the axial plane. Another specific example includes a device 10 movable in just the coronal plane and fixed in the sagittal and axial planes. The specific device and placement within the patient can be selected to achieve the desired type, direction, and extent of dynamic movement.

The device 10 may be positioned at various spinal regions including the cervical, thoracic, and lumbar regions. The device 10 may be used with a variety of constructs 100 used for a variety of spinal treatments including, but is not limited to, treatment of degenerative spondylolisthesis, fracture, dislocation, scoliosis, kyphosis, spinal tumor, PJK, hyperlordosis, hypolordosis, and/or a failed previous fusion. The device 110 may also be used in constructs for other applications. Examples include but are not limited to treatment of a patient's long bones (e.g., femur, tibia, fibula, humerus).

In one embodiment, the anchor 30 is top-loaded into the receiver 20. Specifically, the shaft 32 and head 31 are inserted through the top side 27 of the receiver 20 and moved downward towards the bottom side 26. The shaft 32 is narrow and fits through the neck 81 with the head 31 being wider and maintained in the reservoir 21. In another embodiment, the anchor 30 is bottom-loaded into the receiver 20. The head 31 is inserted through the bottom side 26 of the receiver 26 and moved into the reservoir 21. After this positioning, the narrow neck 81 is formed in the receiver 20. This formation may include deforming the walls of the receiver 20 through a turning operation as disclosed in U.S. patent application Ser. No. 12/038,572 which is herein incorporated by reference in its entirety. The formation may also include securing a member that includes the narrow neck to the receiver 20. In one embodiment, the narrow neck is formed in an annular member that is placed around the anchor shaft 32 and moved over the shaft 32 and to the receiver 20. The annular member is then attached to the receiver 20 using various techniques, such as welding or soldering, or the annular member includes threads that are mated with corresponding threads in the receiver 20.

The device 10 may be used during surgical procedures on living patients. The device 10 may also be used in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A device for attaching an elongated member to a vertebral member comprising:
an anchor having a head and an outwardly-extending shaft;
a receiver attached to the anchor and including a channel configured to receive the elongated member and a reservoir configured to receive the head of the anchor, the receiver including a longitudinal axis that extends through the channel and the reservoir;
the reservoir including an elongated shape with a major axis that extends between opposing first and second ends and a minor axis, the reservoir being larger than the head of the anchor with the receiver movable about the head at a plurality of positions;
a first dampener positioned in the reservoir between the first end and the head of the anchor to dampen movement of the receiver relative to the anchor in a first direction; and
a second dampener positioned in the reservoir between the second end and the head of the anchor to dampen movement of the receiver relative to the anchor in an opposing second direction, the second dampener being separate from and spaced away from the first dampener.

2. The device of claim 1, wherein the first dampener includes a first construction and the second dampener includes a different second construction.

3. The device of claim 1, wherein the first dampener includes a greater thickness measured along the major axis than the second dampener.

4. The device of claim 1, wherein a first distance in the reservoir between the longitudinal axis and the first end is greater than a second distance between the longitudinal axis and the second end.

5. The device of claim 1, wherein the reservoir further includes an arced shape in a plane that bisects the channel with a central region of the reservoir being closer to the channel than each of the first and second ends.

6. The device of claim 1, wherein the receiver further includes a neck that forms a reduced opening in communication with the reservoir, the opening having a smaller width than the head of the anchor.

7. The device of claim 1, further comprising a fastener that engages with the receiver and applies a downward force to the elongated member into the channel, the head of the anchor being spaced away and isolated from the channel with the head being isolated from the force.

8. The device of claim 1, wherein the reservoir is sized relative to the head of the anchor for the receiver to be rotatable about the anchor.

9. The device of claim 1, further comprising an opening within an interior of the receiver that extends between the channel and the reservoir, the opening including a smaller width than the head of the anchor.

10. A device for attaching an elongated member to a vertebral member comprising:
an anchor having an enlarged head and an outwardly-extending shaft;
a receiver including a reservoir that receives the head of the anchor with the reservoir being larger than the head of the anchor for the receiver to be movable to a plurality of positions relative to the anchor, the receiver further including a channel configured to receive the elongated member, the receiver including a longitudinal axis that extends through the channel and the reservoir;
the reservoir including an elongated curved shape in a plane that extends through the channel with first and second ends positioned a greater distance from the channel than a central section of the reservoir;
a flexible dampener positioned in the reservoir and positioned between the first end of the reservoir and the head of the anchor, the dampener being positioned to be compressed when the receiver moves in a first direction relative to the anchor.

11. The device of claim 9, further comprising an additional dampener positioned in the reservoir between the second end and the head of the anchor and being positioned to be compressed when the receiver moves in an opposing second direction relative to the anchor, the additional dampener being separate from and spaced away from the dampener.

12. The device of claim 11, wherein the additional dampener includes a different flexibility than the dampener.

13. The device of claim 10, wherein a first distance between the longitudinal axis and the first end of the reservoir is greater than a second distance between the longitudinal axis and the second end of the reservoir.

14. The device of claim 10, wherein the receiver further includes a narrow neck that defines an opening that extends into the reservoir and includes a smaller width than the head of the anchor.

15. The device of claim 10, further comprising a fastener that engages with the receiver and applies a downward force to the elongated member into the channel, the head of the anchor being spaced away from the channel with the head being isolated from the force.

16. The device of claim 10, wherein the reservoir is sized relative to the head of the anchor for the receiver to be rotatable about the anchor.

17. The device of claim 10, further comprising an opening within an interior of the receiver that extends between the channel and the reservoir, the opening including a smaller width than the head of the anchor.

18. A method of stabilizing a patient's spine comprising:
attaching a dynamic device to a vertebral member;
aligning a receiver of the dynamic device relative to the vertebral member with a major axis of a reservoir in the receiver aligned in a sagittal plane of the patient's spine with a first dampener positioned on a first side of an anchor head and a second dampener positioned on a second side of the anchor head, the first and second dampeners being spaced apart and separate;
positioning an elongated member into a channel of the receiver;
attaching a fastener to the receiver and positioning the elongated member against a bottom of the channel with the receiver being movable relative to the anchor; and
the dynamic device configured for the receiver to move relative to the anchor in a first direction in the sagittal plane to compress the first dampener and to move in an opposing second direction in the sagittal plane to compress the second dampener.

19. The method of claim 18, further comprising positioning the dynamic device for the receiver to rotate about the anchor head.

20. The method of claim 18, positioning the dynamic device to limit movement of the receiver in the first direction to about 3-4° and to limit movement of the receiver in the second direction to about 6-8°.
